(19) United States
(12) Patent Application Publication
(10) Pub. No.: US 2019/0333609 A1
(43) Pub. Date: Oct. 31, 2019

(54) Sample preservation container, system for processing information relating to a sample, and sample analysis process

(71) Applicant: FAST-ENGINEERING SPRL, Gembloux (BE)

(72) Inventor: Pie Olivier Atangana, Namur (BE)

(21) Appl. No.: 16/471,749

(22) PCT Filed: Dec. 20, 2017
(86) PCT No.: PCT/EP2017/083900

§ 371 Date: Jun. 20, 2019

(30) Foreign Application Priority Data
Dec. 20, 2016 (BE) 2016/5949

(51) Int. Cl. G16H 10/40; G06F 9/451; G16B 50/00

(52) U.S. Cl. CPC G16H 10/40; G16B 50/00; G06F 9/451

(57) ABSTRACT

The present invention relates to a system for processing information relating to a sample intended for laboratory analysis, said sample being handled as part of a process in which it is transferred from a client to a laboratory via a common carrier. The invention also relates to: a sample analysis process, comprising a step in which the sample is transferred from a client to a laboratory by a common carrier; and a container for receiving and preserving samples, which can be used in a system and process according to the invention. The container comprises: at least one compartment for receiving at least one sample, said compartment comprising a door, a means for locking/unlocking the door and a sample presence detector; and a data transfer device connected to a computer system for processing information relating to the samples.

### TECHNICAL FIELD

The present invention relates to the temporary preservation of biological samples and systems for processing information relative to a sample intended for a laboratory analysis, said sample being handled in a transfer process from a client to a laboratory via a carrier.

### BACKGROUND OF THE INVENTION

A process for performing medical biology exams generally includes three phases: a first pre-analytical phase, a second analytical phase and a third post-analytical phase. The pre-analytical phase generally comprises the steps of requesting analysis of a biological sample in a laboratory, preparation and identification of the patient, collecting the primary sample, preserving it and delivering the sample to an analysis laboratory.

In the pre-analytical process, a doctor may prescribe his patient the collection of a biological sample for the purposes of a medical diagnostic. This sample collection can be done by the doctor himself, by the patient himself, or by a medical center.

Depending on the type of biological sample to be collected, the sample is directly collected by the doctor in his medical office and sent to the laboratory by the doctor or the medical center, or the sample is provided by the patient to the doctor or the medical center, which next handles the sending of the sample to a laboratory.

In all of these cases, the biological sample is generally packaged in a container for a certain length of time while waiting for a transport service to make arrangements to pick up this sample and take it to a laboratory.

Unfortunately, the collection hours of a sample of adequate quality for good analysis (nonlimiting examples: blood, stool, urine) and the availability times for this sample for transport services are always different. Furthermore, the geographical distances between the collection location and the delivery location of the sample are often quite significant. This situation is even more pronounced in extremely large countries that have a substantial rural population and where the medical, road and/or medical analysis infrastructure is limited or nonexistent.

This situation causes major problems for the bio-logistic and quality management organization of the samples before they are delivered to a laboratory. Typically, it is impossible for the laboratory to know, with precision and in advance, when the samples will be available on a remote collection site or in what quantity, without mobilizing substantial and sometimes expensive human resources.

It is also impossible for the laboratory to know the quality and preservation conditions that existed before the samples were collected by a carrier to deliver them to the laboratory. To minimize the gravity of the previously described situation, this currently requires that the laboratories use very substantial and very costly physical means, in particular logistical, but nevertheless without eliminating many quality and logistical problems encountered with the biological samples (example: very costly systematic rounds, lack of control over temperature variations (in summer or winter, etc.)).

Taking all of the constraints into account, this requires mastery of the pre-analytical phase by an interconnected autonomous system allowing a drop-off, preservation in accordance with quality standards, secure pick-up and automated communication between the drop-off point of the samples and the laboratory associated with its logistical service.

Following this observation, there is therefore a major need to optimize the collection, preservation, pick-up method and the necessary tools in the biological sample transfer process in the pre-analytical phase (example: from a medical office, a collection center, to an analysis laboratory).

### BRIEF DESCRIPTION OF THE INVENTION

The present invention relates to a system for processing information associated with a sample intended for a laboratory analysis, said sample being handled in a transfer process from a client to a laboratory via a carrier, as described in the claims. The invention also relates to a process for analyzing a sample comprising a transfer step from a client to a laboratory via a carrier as defined according to the claims. The invention also relates to a container as defined according to the claims.

The present description relates to a sample drop-off and/or preservation container comprising at least one compartment for dropping off at least one sample, said compartment comprising a door, a means for locking and unlocking said door and a sample presence detector, characterized in that said drop-off container comprises a data transfer device connected to a computer system for processing information associated with said samples.

Advantageously, said sample drop-off and/or preservation container comprises a means for reading a code relative to a sample and a means for checking the locking and unlocking of said compartment for the drop-off and pick-up of samples.

Preferably, said sample drop-off and/or pick-up container comprises a device for communicating with at least one user of said container making it possible to communicate the progress status to said user of an identification, drop-off or pick-up operation of a sample.

Preferably, said sample drop-off and/or preservation container comprises at least one means for checking predetermined physical preservation parameters of a sample.

Preferably, said sample drop-off and/or preservation container comprises at least one means for measuring, calculating and checking physical environmental parameters making it possible to adapt the predetermined preservation parameters of a sample based on those of the environment.

Preferably, said drop-off and/or preservation container comprises an electrical power source from the grid.

Preferably, said drop-off and/or preservation container comprises an electrical power source from the grid and a backup battery for emergency power in case the electricity supply from the grid fails.

Preferably, said drop-off and/or preservation container comprises a power source from the grid, a backup battery and an emergency power source in case of power outage on the grid and a photovoltaic or wind device or a combination of the two making it possible on the one hand to recharge the battery and on the other hand to guarantee energy autonomy and continuity of service of the container.

Preferably, said drop-off and/or preservation container is geolocatable.

Preferably, said drop-off and/or preservation container comprises at least one screen for digitally displaying technical and functional information of said container; it comprises at least one light signaling device allowing the container to communicate regarding the status of the access system towards the various users.

Preferably, said drop-off and/or preservation container comprises a digital keyboard, optionally virtual, making it possible to enter information directly.

Preferably, said drop-off and/or preservation container comprises at least one digital screen for displaying medical, time and sensitization information.

Preferably, said sample drop-off and/or preservation container is connected via the Internet to said system for processing information associated with said samples.

Preferably, said sample drop-off and/or preservation container is connected to the Internet via a Wi-Fi connection or a mobile connection and it comprises a system making it possible to switch from one type of connection to the other based on the quality of the Internet throughput.

Preferably, said sample drop-off and/or preservation container interacts with a cloud computing environment including a server, a database and an application, so as to communicate data between said container and said database, said cloud computing environment further interacting with at least one user interface so as to receive data from said interface and transfer it to said database and to send data from said database to said interface, said cloud computing environment and said user interface being part of said system.

The present invention relates to a system for processing information associated with a sample intended for laboratory analysis, said sample being handled in a transfer process from a client to a laboratory via a carrier, which comprises:
a cloud computing environment including a server, a database and an application;
a user interface interacting with said cloud computing environment and making it possible to
   introduce data relative to the sample into a database;
   receive data relative to the sample;
   send a task notice to be performed;
   receive a task notice to be performed;
a sample drop-off and/or preservation container comprising at least one compartment for dropping off at least one sample, said compartment comprising a door, a means for locking and unlocking said door and a sample presence detector, said drop-off container comprising a data transfer device connected to said cloud computing environment.

Said system comprises:
a first user interface allowing said client to
   introduce data into said database relative to a sample;
   send an invitation to a laboratory to perform an analysis of the sample;
   receive a notice of agreement from said laboratory to perform said analysis;
   generate a drop-off code for said sample and a marking of said sample associated with this drop-off code in order to allow the drop-off of said sample in said preservation container;
   receive notices relative to the tracking of said sample and the results of the analysis of said sample;
a second user interface allowing said laboratory to:
   receive said invitation to perform an analysis for said sample;
   send said client a notice of agreement to perform said analysis;
   receive notices relative to the tracking of said sample;
   introduce information into said database relative to the receipt of said sample; and
   communicate the results of the analysis of the sample to said client;
a third user interface allowing said carrier to:
   receive an invitation to pick up a sample in said preservation container;
   obtain a pick-up code for said sample;
   receive the location of said laboratory in which said sample must be delivered.

Advantageously, the data relative to physical preservation conditions of the sample in said drop-off container, predetermined as a function of the nature of said sample, is stored in said database, and used in an application communicating with said container such that a control means regulates said physical preservation conditions when a sample is located in one said compartment.

Advantageously, the data relative to temporal preservation conditions of the sample in said drop-off container, predetermined as a function of the nature of said sample, is recorded in said database, and used in an application communicating with said container, such that a detector detects the moment at which said sample is introduced into said container and communicates said moment to said application, which sends to an interface used by said carrier an invitation to pick up said sample and deliver it to said laboratory in a predetermined time frame.

Preferably, the data relative to the preservation of said sample is recorded in said database, and communicated to an interface used by said laboratory.

Preferably, the data relative to the transport of the sample is recorded in said database via an interface used by said carrier and communicated to said client and said laboratory.

Preferably, said system comprises:
a first interface allowing said client to encode its identification data and/or that of the patient or to import all of said data automatically, to generate and communicate a request for analysis of a sample to a laboratory directly, to generate an identification code for drop-off of said sample in order to allow the drop-off of said sample in a drop-off container according to any one of the preceding paragraphs, to generate and send a notice or a code requesting pick-up of said sample intended for said carrier and to allow the client to receive notices relative to the tracking of said sample and the results of the analysis of said sample;
A second interface allowing a client to connect to third-party databases, whether they are public or private, and to download therefrom or record therein medical data of its patients;
A third interface allowing a laboratory to receive said analysis request; to receive said sample; to check the quality of said sample; to receive notices relative to the tracking of said sample and to communicate the results of the analysis of said sample to said client and said patient;
A fourth interface allowing a carrier to receive:
   a drop-off notice of a sample in said drop-off container at said predetermined location, which is geolocatable, and an invitation to go pick up said sample from said container;
   an activation right of an access code or a badge comprising this access code to said container and to said compartment;
   said pick-up code for said sample;
   the address of said laboratory where said sample must be delivered;
A fifth interface allowing a patient to receive:
   A notice from the laboratory and/or the client,
   A notice regarding the tracking of the analysis of his sample,
   A notice making it possible to receive the results of the analysis of the sample,
A sixth interface allowing an administrator of the system to have remote access and control of all of the containers and interfaces; to receive technical and operating notices for the containers; to receive notices from all of the users and interfaces.

Advantageously, the operation for generating an analysis request for a sample includes the introduction of characteristics of the sample that determine physical characteristics for preservation of the sample in said drop-off container, and in which the drop-off operation of said sample in said drop-off container triggers a means for controlling physical preservation parameters of said sample so as to preserve said sample under predetermined physical conditions as a function of the type of sample. Preferably, said system operates independently of the operation for generating a request to analyze the sample including the introduction of characteristics of the sample to determine physical preservation conditions of the sample in said drop-off container.

Preferably, the operation for generating an analysis request for a sample includes introducing characteristics of the sample to determine a preservation time frame of said sample, and in which a preservation and pick-up time frame of said sample is notified to said client, said carrier and said laboratory.

Preferably, said system establishes statistics on at least one or several of the following elements: the accounting of generated analysis requests; sample drop-offs; sample pick-ups, sample deliveries, usage and access data to all of the interfaces by all of the users; the operating statuses of the containers.

Preferably, said system automatically generates a quality report covering the entire process as of delivery of the sample to the laboratory and wherein said quality report is done in accordance with said physical and preservation time conditions having respectively been respected or not.

The present invention also relates to a process for analyzing a sample comprising a transfer step from a client to a laboratory via a carrier, characterized in that it comprises a step for dropping off said sample in a sample drop-off and/or preservation container comprising at least one compartment for dropping off at least one sample, said compartment comprising a door, a locking and unlocking means of said door and a sample presence detector, characterized in that said drop-off container comprises a data transfer device connected to a computer system for processing information associated with said samples.

Advantageously, said carrier receives, via said system, an invitation to pick up the sample from said container and deliver said sample to said laboratory.

Preferably, the leasing laboratory receives said sample and validates the receipt of the sample after a step for verifying that the preservation conditions of said sample and transport conditions of said sample have been respected.

Preferably, the quantitative logistical data is sent to the laboratory regarding the specific type and quantity of samples having to be delivered to it; the production planning data regarding the delivery time frames of the samples; all of this data is dynamic and is sent to the laboratory in real time.

According to one preferred embodiment, the information processing system according to the invention is characterized in that it comprises:
a cloud computing environment including a server, a database and an application;
a sample drop-off and preservation container comprising at least one compartment for dropping off at least one sample, said compartment comprising a door, a means for locking and unlocking said door and a sample presence detector, said drop-off container comprises a data transfer device connected to said cloud computing environment;
several user interfaces interacting with said cloud computing environment and making it possible to
   introduce data relative to the sample into a database, based on data which are pre-existing, non-active and strictly not previously assigned to a sample;
   receive data relative to the sample based on introduced, pre-existing, activated data singularly assigned to a sample in said database;
   send a task notice to be performed based on introduced, activated data singularly assigned to a sample in said database;
   receive a task notice to be performed based on introduced, activated data singularly assigned to a sample in said database;
      said interfaces comprising:
a first user interface allowing said client to:
   introduce, into said database, data relative to a sample based on pre-existing, nonactive data strictly not previously assigned to a sample;
   access nonactive and pre-existing data in said database, activate the use of introduced, pre-existing data to assign it singularly to a sample and specifically to a laboratory;
   send an invitation to a laboratory to perform an analysis of a sample based on introduced, activated data assigned singularly to a sample in said database;
   receive a notice of agreement from said laboratory to perform said analysis based on introduced, activated data assigned singularly to a sample in said database;
   activate a pre-existing drop-off code in said database and assigned singularly to said sample in order to allow the drop-off of said sample in said preservation container;
   receive notices relative to the tracking of said sample and the analysis results of said sample based on introduced, activated data assigned singularly to said sample and validated by said laboratory, said carrier in said database;
a second user interface allowing said laboratory to:
   introduce, in a database, nonactive data strictly not previously assigned to a sample in said database;
   singularly assign the use of said nonactivated data to a specific client so as to allow the use, the access and the activation of said nonactive data solely to said specific client;
   receive said invitation to perform an analysis for said sample based on the introduced, activated data singularly assigned to said sample in said database;
   send said client a notice of agreement to perform said analysis based on the introduced, activated data singularly assigned to said sample and validated in said database;
   receive notices relative to the tracking of said sample based on the introduced, activated data singularly assigned to said sample, said client and said carrier in said database;
   introduce information into said database relative to the receipt of said sample based on the introduced, activated data singularly assigned to said sample in said laboratory in said database; and
   communicate, to said client, the analysis results of the sample based on the introduced, activated, validated data assigned specifically to said laboratory by said client;
a third user interface allowing said carrier to:
   receive an invitation to go pick up a sample in said preservation container based on a pre-existing pick-up code, activated in said database and assigned singularly to said sample and to said laboratory;
   activate a pick-up code for said sample based on an invitation to go pick up a sample in said preservation container;
   receive the location of said preservation container in which said sample must be picked up and delivered to said laboratory.

### BRIEF DESCRIPTION OF THE FIGURES

FIG. 1 shows a view of a drop-off container according to one embodiment of the invention.

FIG. 2 shows a status diagram of the operations in an analysis process according to the present invention.

### DETAILED DESCRIPTION OF THE INVENTION

According to a first aspect, the present invention relates to a sample drop-off and preservation container, which is usable in an information processing system in a process according to the invention. The samples can be biological or chemical samples. Preferably, the samples are biological samples obtained after collection by a doctor or a medical center in the case of nonlimiting examples of the present invention of a blood draw, stool and/or urine collection, Pap smear, biopsy, or by the patient himself in the case of nonlimiting examples of the present invention of stool or urine collection.

The sample drop-off and preservation container comprises at least one compartment for the drop-off and preservation of a sample, in a step of a process for transferring a sample from a client to a laboratory via a carrier. According to one preferred embodiment, the container comprises:
At least one door allowing access to each of the compartments, i.e., each compartment can have one or several doors, preferably two: one door that allows only the drop-off of samples in the compartment but not the collection of samples, and one door that allows the collection of one or several samples, preferably a packet that comprises several samples. The drop-off door can take the form of a window provided in the collection door. In another embodiment, a compartment can have a single door that serves as drop-off door and collection door. The doors are provided with means for locking and unlocking said doors,
at least one means for checking said locking and unlocking,
at least one means for checking one or several physical parameters inside the compartments, for example the temperature,
at least one means for detecting a code associated with one or several samples. This involves a code reader such as a barcode reader. The reader is connected to the means for checking the locking/unlocking of the doors such that the reading of a code activates the unlocking of a door, allowing the access to a compartment, to drop off a sample or to pick up one or several samples,
preferably, a means for detecting the presence of samples in the compartment(s),
a compartment comprising a plurality of electronic elements configured to implement said detection, control and data transfer means,
a power source for said electronic elements and/or a connection that makes it possible to power said elements via an external source.

Furthermore, the container comprises a data transfer means connected to a computer system for processing information associated with said samples. According to one preferred embodiment, the container is characterized in that it interacts with a cloud computing environment including at least a server, a database and an application, so as to communicate data between said container and said database, said cloud computing environment further interacting with at least one user interface so as to receive data from said interface and transfer it to said database and send said data from said database to said interface.

According to one preferred embodiment, the container serves as an autonomous receptacle for the temporary drop-off and preservation of samples, the container being configured to operate in environments with uncontrolled temperatures. The containers according to the invention can be installed in different locations and under different and variable ambient conditions. Their function is to receive, in a controlled manner, a sample deposited by a client (e.g., doctor, patient) and to keep it under controlled conditions (in particular under a controlled temperature, even when the ambient temperature varies).

The term “client” can apply to a doctor or a patient or even in general a user involved in a step for depositing a sample in the container.

The control and detection means will be described in more detail below. These control and detection means can interact directly with the cloud computing environment. According to one embodiment of the invention, the preservation container can also comprise a local processor and a memory interacting with the cloud computing environment. In this case, control means and detection means can interact indirectly with the cloud computing environment via said local processor.

The sample preservation container according to the invention can be considered a smart container, allowing doctors or medical centers to receive medical samples in accordance with the preservation and safety constraints of the samples. Furthermore, both the recipient and the delivery agent have the possibility of tracking the status of the sending of the samples.

Preferably, said container comprises a plurality of compartments.

Each of the compartments comprises at least one door and a means for locking said door(s).

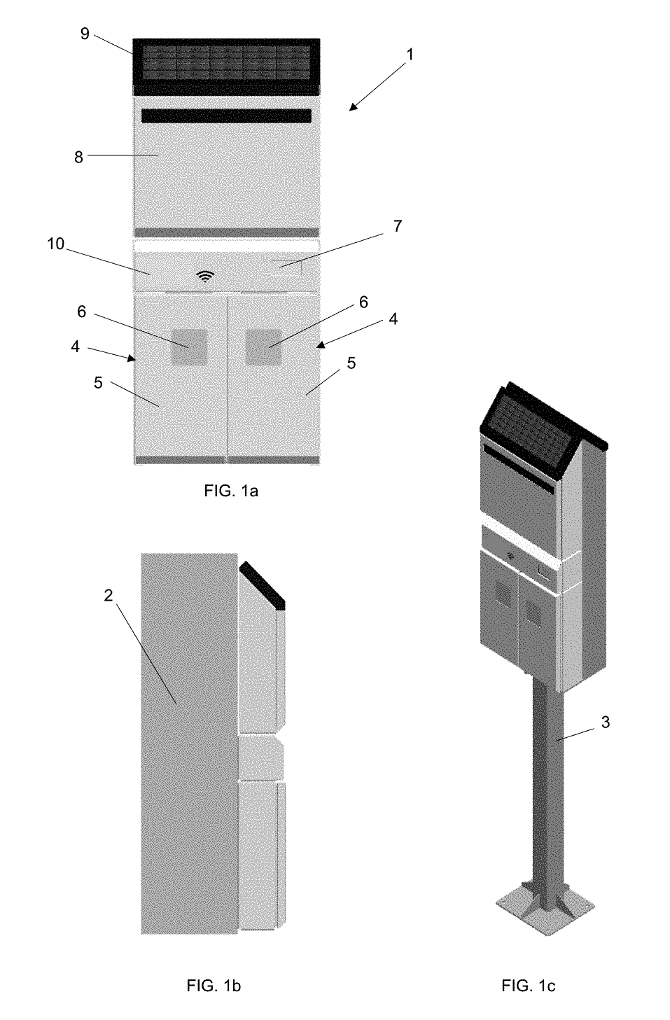

FIG. 1a shows a container 1 according to one embodiment of the invention. The container can be attached to a wall 2 (FIG. 1b) or installed on a support leg 3 (FIG. 1c). The container comprises two compartments 4, each compartment comprising its own sample collection door 5 that opens by tilting about a rotation axis below the door 5. In each of the doors 5, a drop-off window 6 is provided. The doors 5 and 6 are equipped with a locking/unlocking system. The collection doors 5 cannot be opened by a party dropping off a sample, but only by authorized persons such as a sample carrier coming to collect the samples or a mechanic to perform upkeep or repairs. The access to the doors 5 and the windows 6 is controlled automatically, by means of a code reader. The container can comprise a compartment 8 for dropping off paper documents. Preferably, the container is provided with a solar panel 9 and battery (not shown), to guarantee the autonomy of the container. A compartment 10 comprises the electronic elements necessary for the operation of the container, such as a reader, a wireless communication module (e.g., Wi-Fi), a location module (e.g., GPS), one or several control units. The container also comprises a screen and a keyboard or a touch-sensitive screen 7 to allow the client to enter data and to allow the container to display data or information.

According to one embodiment, the container is provided with a facial recognition system for a user, allowing automatic identification of authorized individuals.

According to one embodiment, the container is configured to communicate with several other containers, preferably containers of the same type, in a meshed network. This makes it possible to communicate, among the containers, the availability to receive samples. A client can for example be faced with a container that no longer has any capacity. Through the network, the container can check the availability of other containers and use its screen to communicate the position of the other container to the client. The meshed network can be produced using technical means known in the state of the art.

The samples intended to be kept in a container according to the invention are preferably marked by an identification code, for example a numerical code, or barcode or QR code, or an RFID tag, or any other marking means allowing an identification of a sample using a detection means (the reader 7) arranged on said container and allowing the reading of the identification code. The reader 7 can be a scanner or a numerical pad or a camera, or any other means allowing the reading of an identification code. When a sample identification code corresponds to a code previously introduced into the database of the cloud computing environment, a control means allowing the opening and closing of at least one door is activated to allow the sample to be introduced into a compartment of the container.

The control means allowing the opening and closing of at least one door can open said access door toward the inside of the container, and a door of a compartment dedicated to the preservation of a sample. The control means allowing the opening and the closing of at least one door is configured to open the door(s) after the reading of the identification code to allow a user to drop off the sample in a compartment or to remove it from a compartment, and is configured to close the door(s) again within a time frame following the opening of the door(s) so as to leave the user a certain amount of time to drop off or pick up the sample.

The container can further comprise additional detection means for detecting any potential problem with the opening or closing of the door, and to communicate an error message to the user and to the cloud computing environment or to a local processor, if, according to one embodiment of the invention, a local processor is also included in said container. In the case where an object or an incorrect positioning of the sample hinders the closing of a door, a detection means detects a signal and sends it to the cloud computing environment or the local processor so as to send an error message to the user, and to activate the opening and closing control means of the hindered door to allow the unblocking of the door. If the problem persists after at least two consecutive error messages, an unavailability notice of the container or the compartment is sent to the cloud computing environment, which uses an application and an interface to communicate a notice to the client and to a service managing the maintenance of the container or to an administrator managing a system comprising said container. The application can send the client a notice via an interface used by the client to point him toward another sample preservation container.

Each compartment of the container can comprise a means for detecting the presence of a sample, this detection means interacting with the cloud computing environment so as to communicate the status of a compartment to an interface used by one or several users. For example, when a container comprises a compartment available for dropping off a sample, this availability status or any other appropriate term appears on a user interface used by the client. The user interface used by a client can for example be a computer or a fixed or mobile telecommunication device. When a sample is introduced into a compartment, the sample presence detecting means communicates a “sample pick-up pending” status that is next communicated to a user interface used by a carrier, for example a mobile telecommunication apparatus. This “sample pick-up pending” status can also be communicated to an interface used by the client and an interface used by the laboratory.

Some samples, depending on their nature, may require special preservation conditions. These special preservation conditions of the samples depending on their nature can be predetermined and recorded in an environment of the cloud computing database. When a sample is intended to be analyzed, data relative to the nature of the sample is introduced via an interface used by the client, preferably the doctor, in the database of the cloud computing environment. This data relative to the nature of the sample to be analyzed is compared with the predetermined data and recorded in the database, such that when a sample is introduced into a compartment of the container after having been identified by the identification code reading means, an application activates means for checking preservation parameters of the sample.

The preservation parameters of the sample can be physical, for example a temperature or a range of preservation temperatures, a humidity level or the like. The preservation parameters of the sample can also be temporal or physico-temporal. For example, a sample can have to be kept for a first period of time at a first temperature, and optionally for a second period of time at a second temperature.

Each compartment of the container can comprise a means for detecting physical parameters such as the temperature or the humidity level. This means for detecting physical parameters interacts with the cloud computing environment or with a local processor in the container. Each compartment of the container further comprises a means for checking physical parameters interacting directly with the cloud computing environment or indirectly through a local processor in said container. When data relative to a sample to be analyzed has been introduced into the database of the cloud computing environment, an identification code has been assigned to the sample, this identification code has been read by the code reading means and the sample has been dropped off in the dedicated compartment, the means for detecting physical parameters and the physical parameter control means are activated so as to regulate the physical parameters in said compartment.

The detection means used to detect the presence of the sample in a compartment detects a signal at the moment when said sample is introduced into said container and communicates said signal to an application in the cloud computing environment, which sends an invitation to an interface used by a carrier to go pick up said sample within a predetermined time frame and to deliver it to said laboratory within a predetermined time frame. These time frames can be recalculated depending on geolocation data provided by the interface used by the carrier and as a function of the location of the container or the location of a plurality of containers used and as a function of the location of the laboratory or the location of a plurality of laboratories.

Preferably, the sample preservation container is supplied with electricity by the grid. Preferably, the container can comprise an internal battery and a system making it possible to switch the power supply from the grid to power from a battery in case of outage from the grid.

The sample preservation container is connected to the cloud computing environment via a network, preferably the Internet. The sample preservation container is connected to the Internet via a Wi-Fi connection or any other type of connection, whether mobile or not. The sample preservation container preferably comprises a system making it possible to switch from a Wi-Fi or local connection to a mobile connection of the 3G/4G type or the like or vice versa, depending on the quality of the Internet throughput or in case of failure of one or the other connection.

According to one embodiment of the invention, the design of the container is simple to use and has the fewest outside elements, to minimize the possibilities for vandalism. The container proposes a scanner that allows reading of the drop-off and pick-up barcode in order to access the container or read an RFID tag, QR code, barcode or any other identification means.

According to one embodiment, the container has two lighted indicators:
A first lighted indicator, for example blue or green, to indicate that the operations have taken place correctly (access to the container, drop-off of the sample, etc.)
A second lighted indicator, for example red, to indicate that the operations have not taken place correctly.

According to one embodiment, the container has a plate made up of subunits that will make it possible to preserve the samples until they are picked up.

The container is governed by a set of parameters that characterize it and any corresponding rules:
irrespective of the environment, indoor or outdoor, the system of the container monitors and automatically adapts the temperature inside the container as a function of the type of sample and the temperature outside said container;
the temperature inside the container, which may be between 4° C. and 14° C. or rarely more, or less depending on medical rules and practices in force, is preferably kept fixed;
if there is no pick-up within two hours after drop-off, the temperature for example goes from 14° C. to 4° C. or another temperature depending on the medical rules and practices in force for a long-term preservation mode;
the alert notices are sent to at least one user once the drop-off and/or pick-up time frames become critical;
the system generates and saves a tamperproof pre-analytical report for each prescription, drop-off, pick-up, delivery of a sample to the laboratory;
the pre-analytical report contains all of the data regarding the sample over the entire pre-analytical process, whether qualitative, logistical, technical or other;
when empty, i.e., when there is no sample in the container, the internal temperature is for example kept at about 14° C. if the temperature outside the container is strictly above the maximum of the temperature inside said container;
when it is empty, i.e., when there is no sample in the container, the internal temperature is for example kept at about 4° C. if the temperature outside the container is strictly below the minimum temperature inside said container;
for long-term storage of no more than 24 hours, the temperature for example goes to 4° C., according to the medical rules and practices in force (less energy-consuming);
the energy consumption that will specify the electricity consumed by the container;
the humidity level that will specify the humidity recorded in the container;
the filling level that will express the quantity of subunits used: e.g., 33% (⅓ of the subunits);
the connectivity, which will express the communication mode used by the container: e.g., Wi-Fi, 3G; based on the signal status, the communication mode may be switched;
the timer of the drop-off, which will express the maximum duration allowed to drop off a sample: e.g., 5 seconds, when there is no more movement; the container opens 1×/sample type (1× for stool, 1× for urine, etc.);
the pick-up timer, which will express the maximum authorized duration to remove the samples from a subunit: e.g., 10 seconds/subunit;
the number of scan attempts to access the drop-off: e.g., 5.

The container may be equipped with a memory allowing local saving of a certain number of codes, and allowing optimal operation even in case of a loss of connection with the cloud computing environment; once the connection is established, the container must be able to synchronize itself automatically to save, without losses, all of the codes and data in the cloud computing environment.

The container can for example be equipped with a scanner reading barcodes, QR codes or the like, on the outside and one on the inside.

The container can have a design that allows a unique manner of placing the sample. One system of subunits per laboratory can be proposed.

The container can be equipped with internal and external temperature sensors and a thermostat. It can also be equipped with a continuous humidity measurement sensor.

As an example, at least three preservation modes of the container can be used:
a standard mode: for example 14° C. for 2 H of preservation;
an economy mode: for example 4° C. up to 24 H of preservation;
a critical mode: for example 1° C. up to 36 H of preservation.

The samples should ideally spend less than 24 H in the container.

The system will take the container from standard mode to economy mode by adapting the temperature range configured as a function of the limit preservation conditions of the samples and as a function of the temperature outside said container. This transition time will be calculated as a function of the inside and outside temperature of the container, and preferably as follows:
if the outside temperature is 5° C., the container stays within its set preservation ranges (equal to plus or minus 1° C.);
if the outside temperature is 0° C. or lower, the container will activate internal heating to reach the temperature of 4° C., then maintain this temperature with a precision of plus or minus 1° C.;
if the outside temperature is strictly greater than 14° C., the container will activate internal cooling to reach 13° C. with a precision of plus or minus 1° C.

Depending on the quality of the Internet throughput, the communication mode of the container can be switched from a Wi-Fi wireless connection to a 3G mobile connection, and vice versa. Likewise for the power supply, in case of electricity outage, the power source must switch to an internal battery.

The drop-off or pick-up of a sample in the container must be done within a fixed timing (for example 5 seconds for drop-off, 10 seconds/compartment for pick-up). Upon expiration of this timing, the container closes automatically to retain the preservation conditions.

The container can enter standby mode to save energy. When the container is empty, the temperature can for example be set at 14° C.

The container can for example have the following states:
Subunit 1 empty or Subunit 2 empty: in this state, the subunit(s) of the container are empty and the subunit(s) of said container do not contain any sample. This means that all of the sample drop-off codes linked to the doctor using the container are deactivated;
this means that all of the codes and/or badges for accessing the empty subunits to pick up samples intended for the laboratories leasing said container are deactivated;
this also means that all of the codes and/or badges for access to the empty subunits by the carrier assigned to pick up samples from said subunit are deactivated.
drop-off pending: in this state, the container is awaiting drop-off of at least one sample either by the patient or by the user doctor, or by any other user authorized to use it.
pick-up pending: in this state, at least one subunit of the container contains at least one sample and the container is awaiting pick-up of at least one sample; the container is not empty; in other words, there is at least one drop-off code connected to the user doctor of the container that is activated;
There is at least one pick-up code or an access badge for said container connected to a leasing laboratory;
At least one pick-up code and access badge for said container is activated and ready to be used by the carrier assigned to perform the pick-up and delivery of this sample to the laboratory.
full: in this state, all of the subunits of the container are 100% full.
This means that said full container can no longer accept a new drop-off until it is emptied;
This means that the samples that are in said container must be removed urgently by the assigned carriers.
error: in this state, the container is not functional due to an anomaly that has occurred and a maintenance operation is required;
maintenance: in this state, the container is not functional and is undergoing maintenance or repair, and during this state, no sample is present or preserved in said container.

There are three possible access code types for the container:
the access code to drop off a sample, called drop-off code: this drop-off code comprises three states:
   created: in this state, the drop-off code has been created by the doctor or a third party entitled to a prescription or the right to handle the samples;
   This means that the drop-off in said container can be done by the patient himself and for all of the samples he was able to collect himself as well;
   This means that the sample drop-off in said container can be done by the doctor himself or any other person authorized to handle and/or drop-off the patient's sample in said container;
   activated: in this state, the analysis form has been sent to a laboratory for a patient and the drop-off code has been activated by the doctor for this patient;
   deactivated: in this state, the patient/the doctor has dropped off the sample in a subunit of the container or the authorized time frame for drop-off of the sample has elapsed.
the access code to pick up a sample, called pick-up code, which comprises three states:
   created: in this state, the pick-up code has been created by an administrator for a carrier;
   activated: in this state, an analysis has been requested from a laboratory by a doctor for the patient;
   deactivated: in this state, the carrier has picked up the patient's sample for which an analysis has been requested by a doctor, or the code is modified or canceled (in case of loss or theft, for example).
the access code to repair or maintain the container, called maintenance code, which comprises two states:
   activated: in this state, the maintenance code has been created by an administrator for a technician and maintenance of the container must be done;
   deactivated: in this state, the technician has perform the maintenance of the container within the allotted time, or the maintenance time frame has elapsed.

According to a second aspect, the present invention relates to a system for processing information associated with a sample intended for laboratory analysis, said sample being handled in a transfer process from a client (e.g., doctor or patient) to a laboratory via a carrier, characterized in that it comprises:
a cloud computing environment including a server, a database and an application (preferably software operating on the server);
several user interfaces interacting with said cloud computing environment and making it possible to
   introduce data relative to the sample into a database;
   receive data relative to the sample;
   send a task notice to be performed;
   receive a task notice to be performed;
one or several sample preservation containers comprising at least one compartment for the preservation of a sample in a sample transfer process step from a client to a laboratory via a carrier, the container comprising:
   at least one door,
   at least one control means, and
   at least one detection means,
the container interacting with said cloud computing environment so as to communicate data between said container and said database.

The system comprises:
a first user interface allowing said client to
   introduce data relative to a sample into said database;
   send an invitation to a laboratory to perform an analysis of the sample,
   receive a notice of agreement from said laboratory to perform said analysis;
   generate a drop-off code for said sample and a marking of said sample associated with this drop-off code in order to allow the drop-off of said sample in said preservation container,
   receive notices relative to the tracking of said sample and the results of the analysis of said sample;
a second user interface allowing said laboratory to:
   receive said invitation to perform an analysis for said sample;
   send said client a notice of agreement to perform said analysis;
   receive notices relative to the tracking of said sample;
   introduce information relative to the receipt of said sample into said database; and
   communicate the results of the analysis of the sample to said client;
a third user interface allowing said carrier to:
   receive an invitation to go pick up a sample in said preservation container;
   obtain a pick-up code for said sample;
   receive the location of said laboratory where said sample must be delivered.

Preferably, the system comprises a fourth user interface used by a maintenance service of the system. This fourth user interface can be configured to receive an error message relative to a preservation container, so as to notify a technician that a maintenance operation on a container is necessary.

Preferably, data relative to physical preservation conditions of the sample in said drop-off container, predetermined as a function of the nature of said sample, is recorded in said database, and used in an application communicating with said container such that a control means regulates said physical preservation conditions when a sample is located in said compartment.

Preferably, data relative to temporal preservation conditions of the sample in said drop-off container, predetermined as a function of the nature of said sample, is recorded in said database, and used in an application communicating with said container, such that a detector detects the moment at which said sample is introduced into said container and communicates this moment to said application, which sends to an interface used by said carrier an invitation to pick up said sample and to deliver it to said laboratory within a predetermined time frame.

Preferably, data relative to the preservation of said sample is recorded in said database, and communicated to an interface used by said laboratory.

Preferably, data relative to the transport of the sample is recorded in said database via an interface used by said carrier and communicated to said client and said laboratory.

The system fits into the process for carrying out medical biological exams, which includes 3 steps: the pre-analytical phase, the analytical phase and the post-analytical phase. The system primarily relates to the pre-analytical phase, which includes:
requesting exams,
preparing and identifying the patient,
collecting the primary sample, for example the discrete part of a bodily fluid, breath, a hair or a tissue collected for test, study or analysis purposes of one or several magnitudes or properties to determine the nature of the whole,
conveying the sample to the container,
preserving the sample temporarily in the container,
picking up the sample,
conveying the sample to the laboratory before the analysis.

The system implements the following scenario:
the doctor prescribes an analysis request for his patient and provides him with a tagged receipt having at least one barcode, QR code or the like;
the patient fills his receptacle and presents it in front of the barcode reader of the container to drop it off. The container verifies the barcode and takes this patient's sample and preserves it under good conditions;
the carrier looks for all of the samples that are assigned to the laboratory for analysis and conveys them to the laboratory;
the laboratory agent checks the admissibility (quantitative and qualitative) of the samples;
the laboratory may optionally institute a time delay based on the number of samples expected and the expiration date to optimize turnarounds (or by communicating the exact desired delivery time to a sample transport service provider);
notices are sent by the system:
   to the doctor to notify him of tracking of the requested samples
   to the patient to notify him to drop off his sample by the due date
   to the laboratory/carrier to notify it that samples for analysis are ready to be picked up
   to the patient to inform him that the results of his sample analyses are available from his doctor and that he can contact the latter for his diagnosis
   to the patient to inform him that the doctor has made changes to his information (e.g.: changes to office hours, office closure dates, etc.)
traceability of all events such as access to the system, access to the container (drop-off time, pick-up time) as well as preservation conditions of the samples is guaranteed in order to guarantee quality of the analyses.

The system has many advantages for all users in the field of medical biological analyses:
the system can make it possible to develop the activities of contractual laboratories by allowing them to spread out containers close to doctors outside their usual working area and thus to multiply analysis operations (and increase their revenue);
the improved traceability and real-time notifications can make it possible to limit the costs inherent to rejected samples (noncompliance with preservation conditions or time frames);
the patient can have an easy and close method of dropping off his samples rather than going to the laboratory, which provides value-added for the patient, whose doctor can take advantage of this to retain clients and justify the investment in the container, whether purchased or leased;
the system can offer better tracking of the analysis process, in which:
   the doctor can track the journey of analysis requests prescribed for his patients;
   the laboratory can be notified of analysis requests sent to it once doctors submit their requests to it, which can allow the laboratory to optimize its sample collection and facilitate planning as well as analysis requests;
   the patient can be notified of the tracking of the analysis of his sample and the availability of his results;
the system can be an effective communication tool for all users; from the doctor to his patient, from the doctor to the laboratory, from the laboratory to the patient, from the system to the technician for the container, from the administrator to the system;
the system can offer traceability automation and comply with rules, for example those imposed by standard ISO15189;
the system can trace all events and the information requested by standard ISO15189 and ultimately make it possible to generate a traceability report;
the system can allow secure energy management according to the configuration of the container;
the system can allow the laboratory to have a comprehensive and real-time view of the volume of samples and analyses being prepared with doctors and the laboratory and thus allow better planning in a previewed manner, of all analyses and organizational operations.

According to a third aspect, the present invention relates to a process for analyzing a sample comprising a step for transfer from a client to a laboratory via a carrier, characterized in that it comprises a step for dropping off said sample in a sample preservation container comprising at least one compartment for preserving a sample, the container comprising a door, at least one control means and at least one detection means, and interacting with a cloud computing environment including a server, database and application, so as to communicate data between said container and said database, said cloud computing environment further interacting with several user interfaces so as to receive data from said interfaces and transfer it to said database and to send data from said database to said interfaces.

Preferably, said carrier receives an invitation to pick up said sample from said container and to deliver said sample to said laboratory.

Preferably, the laboratory receives said sample and validates the receipt of the sample after a step for verifying that preservation conditions of said sample and transport of said sample have been respected.

According to one embodiment of the process according to the invention, the process for analysis of the samples is initiated by a step for creating the prescription form by the doctor for a patient. The collection being done, the patient/the doctor drops off the collected sample in the container. The carrier notified of an analysis request picks up the sample to conduct the analysis. The sample previously being received and verified by the laboratory agent, is analyzed by the laboratory. The results are sent to the doctor and the patient is notified that his results are available from his doctor. The traceability report is then created and available at the authorized laboratory. The pre-analytical phase stops when the system is in the “Analysis pending” state. The post-analytical phase will begin when the system is in the “Analysis results sent” state and will end in the “Analysis request archived” state.

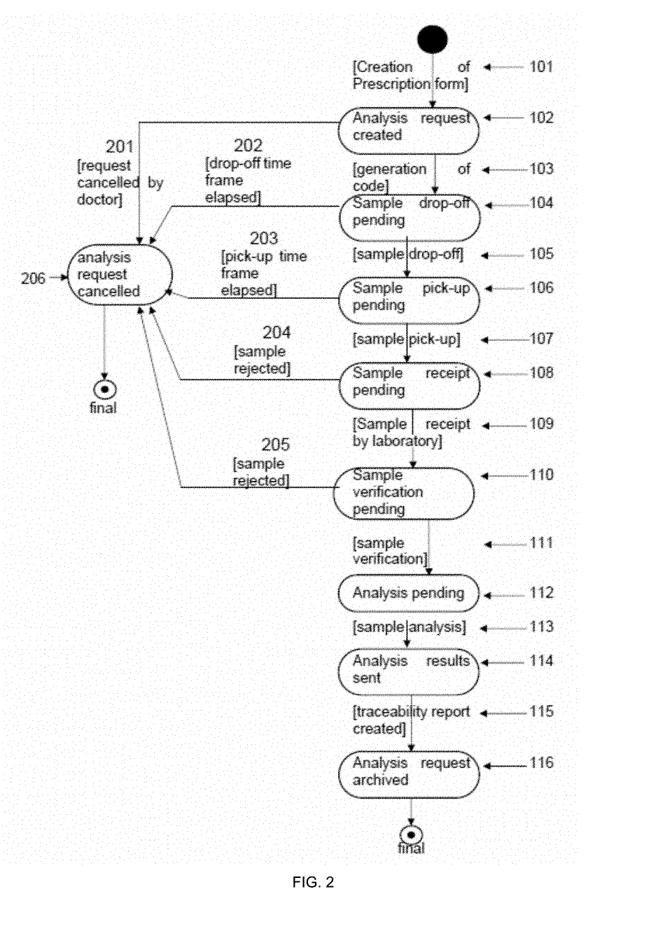

FIG. 2 shows a detailed view of the states of the sample analysis process. The sample analysis process is initiated by a first step 101 for the creation of the prescription form by the doctor for a patient. The process is in a state 102 in which an analysis request is created. In this state, a step 103 for generating a drop-off code is created by a doctor for a patient. A state 104 follows for the container pending sample drop-off and a step 105 for drop-off of the sample in the container within a time frame set by the patient or alternatively by the doctor. This is followed by a state 106 of the container pending pick-up of samples and a step 107 in which a carrier must pick up the sample within a set time frame. This is followed by a state 108 of the method pending receipt of samples and a step 109 for receipt of samples conveyed by the carrier to the laboratory. Next is a state 110 of the method pending verification of samples and a step 111 in which the sample is verified (quantitatively and qualitatively) by a laboratory agent with the assistance of a quality report. This is followed by a state 112 of the process pending analysis and a step 113 for analysis and sending of an analysis report to the doctor. This is followed by a step 114 of the process “Analysis results sent” and a step 115 for creation of a traceability report. At the end 116 of the process, the analysis request is archived with the pre-analytical report comprising the traceability report including all of the conformities and nonconformities and the quality report.

The process can be found in a state 206 in which the analysis request is canceled if:
an event 201 occurs in which the request is canceled by the doctor for any reason; or
an event 202 occurs in which the sample has not been dropped off in the container within the allotted drop-off time frame; or
an event 203 occurs in which the sample has not been picked up from the container within the allotted pick-up time frame; or
an event 204 occurs in which the sample is rejected during the state 108 pending receipt of the sample, for any reason such as an exceeded delivery time frame; or
an event 205 occurs in which the sample is rejected during the state 110 of the method pending verification of the sample, for a reason of nonconformity of the sample;
   and the analysis process of the sample is stopped.

1. A system for processing information associated with a sample intended for laboratory analysis, said sample being handled in a transfer process from a client to a laboratory via a carrier, the system comprising:
a cloud computing environment including a server, a database and an application;
a sample drop-off and preservation container comprising at least one compartment for dropping off at least one sample, said compartment comprising a door, a means for locking and unlocking said door and a sample presence detector, and a data transfer device connected to said cloud computing environment;
several user interfaces interacting with said cloud computing environment and making it possible to
introduce data relative to the sample into a database;
receive data relative to the sample;
send a task notice to be performed;
receive a task notice to be performed;
said interfaces comprising:
a first user interface allowing said client to
introduce data into said database relative to a sample;
send an invitation to a laboratory to perform an analysis of the sample;
receive a notice of agreement from said laboratory to perform said analysis;
generate a drop-off code for said sample and a marking of said sample associated with this drop-off code in order to allow the drop-off of said sample in said preservation container;
receive notices relative to the tracking of said sample and the results of the analysis of said sample;
a second user interface allowing said laboratory to:
receive said invitation to perform an analysis for said sample;
send said client a notice of agreement to perform said analysis;
receive notices relative to the tracking of said sample;
introduce information into said database relative to the receipt of said sample; and
communicate the results of the analysis of the sample to said client;
a third user interface allowing said carrier to:
receive an invitation to pick up a sample in said preservation container;
obtain a pick-up code for said sample;
receive the location of said laboratory in which said sample must be delivered.

2. The system according to claim 1, wherein the system allows to:
record, in said database, data relative to physical preservation conditions of the sample in said drop-off container, predetermined as a function of the nature of said sample, and
use the recorded data in an application communicating with said container such that a control means regulates said physical preservation conditions when a sample is located in one said compartment.

3. The system according to claim 1, wherein the system allows to:
record, in said database, data relative to temporal preservation conditions of the sample in said drop-off container, predetermined as a function of the nature of said sample, and
use the recorded data in an application communicating with said container, such that a detector detects the moment at which said sample is introduced into said container and communicates said moment to said application, which sends to an interface used by said carrier an invitation to pick up said sample and to deliver it to said laboratory within a predetermined time period.

4. The system according to claim 1, wherein data relative to the preservation of said sample is recorded in said database, and communicated to an interface used by said laboratory.

5. The system according to claim 1, wherein data relative to the transport of the sample is recorded in said database via an interface used by said carrier and communicated to said client and said laboratory.

6. The system according to claim 1, wherein it establishes statistics on at least one or several of the following elements: the accounting for generated analysis requests; sample drop-offs; sample pick-ups, sample deliveries, usage data and access to all of the interfaces by all of the users; operating states of the containers.

7. The system according to claim 1, wherein it automatically generates a quality report covering the entire process as of delivery of the sample to the laboratory and said quality report is done according to whether said physical and preservation time frame conditions have respectively been respected.

8. A process for analyzing, using the system according to claim 1, a sample, comprising a transfer step from a client to a laboratory via a carrier, characterized in that it comprises a step for dropping off said sample in a sample drop-off and/or preservation container comprising at least one compartment for dropping off at least one sample, said compartment comprising a door, a locking and unlocking means of said door and a sample presence detector, and a data transfer device connected to a computer system for processing information associated with said samples.

9. The process according to claim 8, wherein said carrier receives, via said system, an invitation to pick up the sample from said container and deliver said sample to said laboratory.

10. The process according to claim 8, wherein the leasing laboratory receives said sample and validates the receipt of the sample after a step of verifying that preservation conditions of said sample and transport conditions of said sample have been respected.

11. The process according to claim 8, wherein the quantitative logistical data is sent to the laboratory regarding the specific type and quantity of samples having to be delivered to it; the production planning data regarding the delivery time frames of the samples; all of this data is dynamic and is sent to the laboratory in real time.

12. A sample drop-off and preservation container usable in a system according to claim 1 to, the container being configured to be installed in a non-temperature-controlled environment, the container comprising:
at least one compartment for the drop-off and preservation of at least one sample, said compartment comprising at least one door,
a means for locking and unlocking said door,
a means for detecting a code associated with one or several samples,
a data transfer means connected to an information processing computer system associated with said samples, and
a means for controlling the preservation temperature of samples preserved in the container.

13. The sample preservation drop-off container according to claim 12, wherein it interacts with a cloud computing environment including a server, a database and an application, so as to communicate between said container and said database, said cloud computing environment further interacting with at least one user interface so as to receive data from said interface and to transfer it to said database and to send data from said database to said interface, said cloud computing environment and said user interface being part of said system.

14. The drop-off container according to claim 12, wherein the container is configured to communicate with several other sample drop-off and preservation containers by a meshed network that includes the container and said other containers.
